(19) United States
(12) Patent Application Publication
(10) Pub. No.: US 2005/0054105 A1
(43) Pub. Date: Mar. 10, 2005

(54) Adenoviral vector system

(76) Inventors: Volker Sandig, Berlin (DE); Ingo Jordan, Berlin (DE)

Correspondence Address: JORDAN AND HAMBURG LLP, 122 EAST 42ND STREET, SUITE 4000, NEW YORK, NY, 10168, US

(21) Appl. No.: 10/491,936

(22) PCT Filed: Oct. 4, 2002
(86) PCT No.: PCT/DE02/03846

(30) Foreign Application Priority Data
Oct. 4, 2001 (DE) 101 50 940.5

(51) Int. Cl. C12N 15/861; C12N 7/00

(52) U.S. Cl. 435/456; 435/235.1

(57) ABSTRACT

The invention relates to an adenoviral vector on the base of human group B adenoviruses, specially of the subtype 11 containing as per invention heterologous elements, inverted terminal repeats (ITRs) in combination with the respective packaging signal of a different serotype virus, preferably of a type B virus. A heterologous promoter, preferably the SV40 promoter is contained and positioned between the packaging signal and the natural position for protein IX in the viral vector. This vector may be additionally deleted in reading frames of the regions E1, E2, E3 or E4. The invention also describes the use of this viral vector for the production of a high capacity vectors based on adenovirus 11, in which the only adenoviral sequences are ITRs and packaging signal and which contains human genomic stuffer sequences. Moreover, cell lines for the amplification of these viral vectors and application of the vectors in medicine are described.

The invention relates to an adenoviral vector on the base of human group B adenoviruses, specially of the subtype 11 containing as per invention heterologous elements, inverted terminal repeats (ITRs) in combination with the respective packaging signal of a virus from different serotype, preferably of a type B virus. A heterologous promoter, preferably the SV40 promoter is contained and positioned between the packaging signal and the natural position for protein IX in the viral vector. This vector may be additionally deleted in reading frames of the E1, E2, E3 or E4 regions. The invention also describes the use of this viral vector for the production of a high capacity vector on the base of adenovirus 11, in which the only adenoviral sequences are ITRs and packaging signal and which contains human genomic stuffer sequences. These heterologous elements in the viral vector on one hand allow stabile propagation in complementing cell lines and on the other hand allow their use as a helper virus for the propagation of a viral vector with stuffer sequences (high capacity vector). Section of the high capacity vector against the helper virus is possible based on heterologous elements. This selection principle may be combined with other selection principles. Moreover, cell lines for amplification of these viral vectors and application of the vectors in medicine are described.

### SCIENTIFIC BACKGROUND

The success of viral Gene Therapy essentially depends on the availability of suitable vectors. The vector is supposed to transport therapeutic genes efficiently into the nucleus of a target cell and bring it to expression, to stabilize it there and is neither toxic nor provokes an immune response. Moreover for broad medical application of a genetherapeutical remedy the vector must be reproducible on industrial scale.

While most of the non-viral vectors fulfil the criteria for production, they are far behind viral systems with respect to their efficiency in spite of intensive research during the recent decade for their application in situ and in vivo.

Adenoviral vectors occupy a special position among the viral systems. They are characterized by a wide host range, by the capability to infect resting cells and by extremely high titres. The infection efficiency related to the necessary amount of nucleic acid exceeds that of all other viral systems and exceeds that of plain DNA 100 000 times (in i.m. application). Types 4 and 7 adenoviruses have been used on a broad scale as live virus vaccines and have a good safety profile. The extremely low integrational tendency of adenoviruses is favourable as an additional safety aspect, since it minimizes the risk of insertion mutagenesis and oncogenic activation. 52 different serotypes of human adenoviruses represent a choice of various viral sheaths with very different tropism. Therefore they are extremely infectious for the liver or muscles (group c), cells of the central nervous system (members of group D) or cells of the hemopoetic system (group B).

Broad spread and therefore a generally high antibody titre in a high percentage of the population oppose the application of vectors of the frequently used serotypes 2 and 5 (group C). It can reduce the efficiency of application even to down to ineffectiveness. However, contamination of the population with viruses of different serotypes varies very much. This means, viruses can be found the capsids of which are only rarely inactivated by antibodies and which show special affinity for certain target tissues. Type 11 adenoviruses, a rare type of the western hemisphere, effectively infects hemopoetic stem cells, dendritic cells and certain tumors. However, so far there is no vector system of this type.

Despite these decisive advantages the application possibilities for adenovirus vectors are limited. This is due to the fact that usual adenoviruses of the first and second generation contain viral genes. Notwithstanding deletion of the most important virus transactivators (E1 region) these genes are expressed in the target tissue. Direct toxicity, cut-off expression, inflammation of the tissue (Simon et al. 1993) and attack of cytotoxic T-lyphocytes are results which finally lead to destruction of infected cells. Additionally there is the hazard of transmission of transforming genes for presently poorly characterized serotypes.

Numerous groups have tried to reduce the immunogenity of adenoviral vectors. E2 and E4 regions, which also have a transactivating function, where eliminated from the virus genome and were transferred into the helper cell line. However, it remains uncertain, whether these changes, which additionally cause reduction of the virus titres, are able to augment the duration of expression in vivo. As consequential continuation of this concept adenovirus vectors were developed in recent years, which are free from viral genes (hardy et al. 1997; Kochanek et al. 1996; Kumar-Singh and Chamberlain 1996; Mitani et al. 1995; Parks et al. 1996). These helper-dependent or high capacity vectors show a significantly altered behavior in animals. Usually typical inflammations following infection of the liver with medium doses of adenoviruses are completely missing and the expression per virus is increased up to 100 times, which allows significant reduction of the dose. Long-term expression up to one year has been proven in various animal models including macaques. Another essential advantage of these vectors is there high packaging capacity. Production of these vectors presently requires co-infection with a helper virus, which supplies the necessary adenoviral proteins in trans. The vectors themselves only contain the non-coding sequences, which are necessary in cis for replication and packaging. This means 35 kb of genomic space remain available. In this way sufficient space for the integration of several genes is left.

Early attempts to establish vectors of this type were little practicable, since all preparations contained great amounts of helper virus, which is difficult to separate and requires a purification in the Cs gradient (Alemany et al. 1997; Mitani et al. 1995). Presently a system dominates, in which the packaging signal of the helper virus in the production cell line is excised by the site-specific recombinase (cre) (Parks et al. 1996, Hardy et al. 1997). After recombination the helper virus still replicates normally, but cannot be packed into virus capsids. The principle allows production of vectors with low helper contamination. The cre recombinase is provided by the production cell line. Alternative to cre the flp recombinase is used. The helper virus itself has to be produced beforehand in cells containing no recombinase.

This system is little robust: as well outgrowth of modified helper viruses as instability of the helper-dependent vector were observed (Sandig et al. 2000). Anyway vectors were produced successfully at laboratory scale. Optimization of both components of the system, helper virus and helper-dependent vector lead to robust production and again enhanced effectiveness in vivo (Sandig et al. 2000). Implementation into large-scale production has remained is difficult. Therefore broad application of this versatile vector system in the correction of metabolic defects, local supply of cytokines, tumor therapy and vaccination is limited. Production begins with the transfection of a helper-dependent vector into the production cell line (293cre), the ITRs of which are either terminal or exist as head-to-head fusion (Parks et al. 1996). Additionally the cells are infected with the adenovirus helper. After obtaining the cytopathic effect the cells are harvested and the vectors liberated by freeze-thaw steps or mild detergents. The cell extract is used for the infection of further passages, which again must be infected with helper virus, if the origin of the innocculum was generated under selective pressure. This process is continued for 5 to 6 passages until sufficient amounts of vector for large-scale infection are generated. The amplification factor is about 20 and lower compared to first generation vectors (30-80). In all presently applied procedures very often helper viruses appear during selection, which escape the selection pressure by mutation and are respectively amplified. They make the vector preparation inapplicable.

So far high capacity vectors were described for group C adenoviruses (types 2 and 5). This is caused by lacking sequence information and the difficulty to produce complete E1 or multiple deleted group B viruses including subtype 11 in a stabile way and with high titres. The vectors mentioned in the patent claims contain specific additional elements stabilizing these vectors and increasing virus titres essentially and moreover enable a new principle of selection against the helper virus.

### CHARACTER OF THE INVENTION

The invention is based on the task to establish a viral vector on the base of the human adenovirus 11. This vector is supposed to be propagable in a stabile way and to be safe in application. Moreover, there is the task to develop robust production methods for such vectors which allow minimize the helper combination.

Establishment of such vector is directed towards limiting vector neutralization by preexisting antibodies and towards the ability to infect new target cells or tissues and therefore to extend the hosts range.

For increasing the safety of the viral vector reading frames of various regions or all viral reading frames must be eliminated.

The task was solved by incorporating heterologous elements into the viral vector.

Such elements are inverted terminal repeats (ITRs) in combination with the respective packaging signal originating from of a different serotype virus, preferably a type B virus. These elements allow an amplification of the virus, but also permit its use as a helper for the production of an adenoviral vector containing stuffer sequences (high capacity vector). They comprise a eterologous promoter, preferably the SV40 promoter, which is positioned between the packaging signal and the natural position for protein IX. Furthermore the task is solved by providing special stuffer sequences for the high capacity vector, which allow stabile amplification of this vector.

By constructing vectors for the first time, which are based on the rare adenovirus subtype 11 in the western hemisphere, inactivation of antibodies is limited. This virus uses infection routs being different from adenovirus subtypes used so far and thus extends the scope of application.

The vectors are used as therapeutics and vaccines.

Vector System Based on the Human Adenovirus Type 11

The following describes the establishment of a vector system for a serotype, adenovirus type 11 being rare in the western hemisphere. Frequency of group C adenoviruses of the types 2 and 5, the usual serotypes in genetherapeutic development and tests, causes high titres of neutralizing antibodies in a large persentage of the population and thus reduces the efficiency of genetherapeutic application. Low distribution of the Ad11 serotype represents a great advantage compared to the used of traditional vectors being based e.g. on Ad5. The nucleic acid sequence of this virus is unknown so far.

Starting point for the vector development was the cloning of the viral genome in a bacterial plasmid. Cloning and sequencing resulted surprising essential differences of Ad11 to the prototypical serotype 5.

(1) The recombinant genome turns out to be surprisingly unstable in the bacterial host cells and appears to be very susceptible to recombination processes. This problem lead to extremely low efficiency in finding a complete genome. Since cloning of such long sequences must apply recombination, this problem was only reduced after successful cloning by selecting suitable bacterial strains for amplifying and purification of plasmid DNA.

(2) Moreover, in the sequences of Inverted Terminal Repeats differ for those of group C viruses and other representatives of group B: Ad11 clearly diverges from other representatives of group B zb Ad7 in the sequence of terminal 22 bp, but is completely identical with the sequences of group C viruses (Ad2 and 5).

Moreover, the ITR of Ad11 has clearly less target sequences for transcription factors as Ad5. Lack of the NF3 binding sequence is very obvious. This cellular factor is involved in the initiation of replications in group C adenoviruses, the covalent activation of the Terminal Protein (pTP) by reaction with desoxycytidinphosphate (dCTP).

This notable difference as opposed to Ad2 and Ad5 possibly explains observations, according to which Ad11 replicates more slowly and limits the scope of permissive cell types beyond the capsid-receptor-interaction. Contrary to Ad5, Ad11 needs a different, possibly less ubiquitous and efficient support by the host cell.

(3) Moreover, in the region between the genes E1B 55K and pIX no TATA box for the expression of the maturation factor pIX was found (see sequence no. 1). These differences open new possibilities for the development of adenoviral vectors, but require modification from traditional constructions.

E1 proteins are eliminated from vectors because of their transforming properties and the transactivating effect on other viral promoters in the vector genome. These proteins or homologues with identical function must be provided by the helper cell. Partially remaining E1 sequences in the vector result in sequence overlaps between helper cell line and vector and this is the cause for development of wild type viruses by homologue recombination. Therefore the E1 sequences have to be completely removed from the vectors. Doing so only 64 bp which have neither binding sites for known transcription factors nor a starting region typical for group C viruses remain before the starting codon of protein IX, which is required for virus packaging. The mechanism for the expression of factor pIX is not clear. In group C viruses this protein is encoded by a specific m-RNA, which is controlled by a separate promoter. Elements within the E1A and B regions might be responsible for transcription of the Ad11 protein IX. Therefore, expression of this open reading frame should be supported by insertion of known heterologous promoter elements, namely the early SV40 promoter. The insertion causes a dramatic increase of the virus propagation. Surprisingly this increase even takes place, when the promoter is hindered to promote the expression of PXI by a directly following poly A signal. This suggests general support by binding sequences of transcription factors, which are found in the SV40 promoter region, for amplification of Adenovirus 11 as a representative of group B2. Only after insertion of this sequence, viruses were rescued after plasmid transfection and amplified with stable unchanged genome. Surprisingly, this effect does not appear when inserted by CMV enhancer sequences in identical position. As invented, a stabilized Ad11 vector is produced by insertion of SV40 promoter sequences. Packaging of adenovirus DNA requires specific interaction between ITR and packaging signal with viral and cellular proteins. The interaction with viral proteins is described as being specific to the subtype (Zhang, 2001) An Ad5 virus with a mutant protein L152/55k cannot pack its DNA and this defect is not complemented by the respective protein of other serotypes. This specificity is most likely mediated by interaction with the packaging signal. A viral Ad11 vector with heterologous Ad5 ITR, but autologous packaging signal therefore ought to be able to replicate due to the identical terminal sequences of the ITR with those from Ad5 and packable due to the Ad11 packaging signal. Surprisingly such vector is not viable. Even more a vector with Ad11 genome and ITR and packaging signal of Ad5 ought not to be viable. However, such vector as described by the invention, is efficiently propagable without additional helper function and Ad11 packaging sequences being absent. It generates a significant surplus of empty virus capsids and therefore is used as a helper virus for a high capacity vector in a special application. This packaging barrier can be combined with other procedures directed towards deletion of cis-active sequences for packaging. Recombination using Cre or flp recombinase are described systems of this type. In this case the heterologous packaging signal is flanked by the recombinase sites and excised or inverted in cells which express the respective recombinase.

Modifications of the vector Ad11 based vector may be used separately. In a preferred embodiment they are inserted together into the vector. In one aspect of the mentioned application in addition to a deletion in E1 in the above described modified Ad11 a deletion in another area of the genome is implemented to generate a second generation Ad11 virus. Possible deletions of this kind are known to the expert. In particular deletions are introduced into the E2B region. Production takes place in a cell which carries the DNA polymerase gene of the C group. Alternatively the polymerase gene of adenovirus type 11 may be expressed in the producer cell. In the first case the gene of the preterminal protein of a C type virus the may be additionally expressed.

Probably an augmented oncogenic potential is located in the E4 region. Therefore, the E4 region of the virus is partly deleted.

In a preferred application the E1 region of Ad11 is deleted and the resulting virus, depends in its replication on trans-complementation by E1 proteins of the host cell.

It is expected that the most effective complementation is mediated by the E1 region of Ad11. Regions E1A and E1B may be established either as separate cassettes, each one fused with heterologous transcription signals (promoter, poly A) or as a unit, connected only with a heterologous promoter of E1A and a heterologous poly A following the reading frame of E1B 55k in the production cell. This requires no or only short overlapping between the sequences in cell line and vector, which would allow no homologous recombination. Such overlapping may have the size between 1 and 6 bp.

It is known that proteins of the E1A region provoke a change of the cell cycle in connection with virus replication. They also activate the transcription without binding directly to DNA, interacting with cellular factors. It is known to those skilled in the art that E1A of a serotype may therefore complement the vector of a different serotype. As is also known, this does not hold true for E1B, because it interacts with other viral proteins, in particular with E4orf6. Therefore, it is not possible to amplify the vector of the invention in the cell line 293, which expresses the E1 region of Ad5. Region E1B codes for two independent proteins of 19k and 55k, which are encoded by a common mRNA. The promoter of E1B is located in the E1A region. It is suggested to express the E1B region either starting from the autologous promoter or from a heterologous promoter. Surprisingly it is found that 55k and 19K expressed as one unit only inadequately complements the vector, irrespective of the fact whether expression happens from a heterologous or autologous promoter. Per invention efficient complementation requires E1B 55k expression separately from E1B 19k by an independent promoter. Inefficiency of the common expression may be due to the fact that the 55k reading frame exists as a second reading frame in a common mRNA and the mechanism for its initiation is inefficient.

High Capacity Vectors

High capacity vectors only require few sequences, which are absolutely necessary for replication and packaging. They cover less than 2% of the size of the genome. Efficient packaging, however, requires a linear DNA molecule covering 75-103% of the wild type genome size flanked by viral terminal repeats (ITR) and containing a packaging signal next to one of the ITRs.

Therefore stuffer sequences are necessary for the production of an efficient vector. They may be of nonviral origin or made synthetically . Selection of these sequences determines the stability of the vector during production as well as its biological impact. If vectors are intended to be used for the long-term expression in the target tissue, it has proven to be advantageous to use of human DNA. It is assumed that this DNA is not recognized by the cell as being foreign and thus is not inactivated. Moreover stabilization in the nucleus could be obtained by means of special sequences interacting with the nuclear matrix (MAR).

The replication mechanism of adenovirus is based on the synthesis of linear DNA molecules starting from protein priming by the terminal protein (TP) and stabilized as single strand DNA as an intermediate. This process supports the pairing of different molecules based on homologous regions resulting in high recombination frequency. Recombination between homologous regions within the stuffer DNA results in heterogeneity of vector populations and potential loss of the transgene. Therefore repetitive sequences in the stuffer DNA must be avoided. This is al strong selection criterion when human DNA is used.

The stuffer DNA moreover should be free from additional, potentially expressible genes. However, gene-free sequences are often heterochromatinized and may enter this state again in the vector. This would limit the expression of the transgene.

In order to solve this contradiction, according to this invention large intronic DNA segments from cellular genes are used. For safety reasons DNA segments free of endogenous retroviruses or elements of these viruses are selected.

For a number of applications it is desirable that this DNA is not recognized as being foreign and no defense reaction is provoked. This is achieved best by using human DNA.

Despite the apparently large amount of available human sequences, the choice of segments meeting the required criteria is limited. Moreover theoretical aspects are not sufficient for finding stable efficiently replicated stuffer sequences showing good gene expression.

A. Sequences of the X Chromosome X152941900-X152976000

Three segments of this region were used in the vector:
1. X152941900- X152951600 fragment 1
2. X152952900- X152963100 fragment 2
3. X152966000- X152976000 fragment 3

Sequences consist of introns of the gene CXorf6 and exons of less than 200 bp, which do not represent an individual open reading frame. They contain no retroviral LTRs. Repeats are limited to short caca, gaga and gtttgttt simple repeats. The GC content of the vector is 47.1% and diverges considerably from the one of adenovirus 5. The replication ability of a vector depends on the properties of its DNA. The underlaying mechanisms were not investigated. It is assumed that vectors with a GC content (GC: content of desoxyguanosin and desoxycytidin nucleotides in the DNA) considerably different from that of adenovirus is inferior in its replication ability to one with an GC content similar to that of adenovirus Following published hypotheses, we expected reduced replication of this vector. Unexpectedly it was found that replication of the vector with a GC much lower then the type 5 adenovirus (55.2%) turns out to be more efficient.

B. In addition sequences of the X chromosome of chrX 149493805-149526200 were selected for a vector. Two segments were determined:
X149495450-X149512868
X1405159901-149526107

The segments contain DNA from the first intron of the FMR2 gene. They are free from retroviral elements and contain only phylogenetically old shine, line and simple repeats, which have diviered far from the respective consensus sequences.

The GC content in the vector is 38.1%. Thus the vector is very suitable for adenovirus types being poor in GC, e.g. those of the genus of AT adenoviruses. The respective sequences were obtained from genomic DNA, extracted from a healthy test person's whole blood by PCR and cloned in vectors which contained ITR as well as packaging sequences of adenoviruses from different serotypes. The DsRed1 gene was included as a transgene. Replication of the vector was compared with existing vectors A and B by co-replication using the Cre-Lox helper system.

A vector's ability for replication depends on the properties of its DNA. Underlying mechanisms were not investigated. It is obvious that a GC content being similar to the one of the adenovirus is inferior in its replicatory efficiency to a vector having a considerably diverging GC content. Surprisingly it is shown here using the described vectors that replication of a vector having a GC considerably under the one of the type 5 adenovirus (55.2%) is more efficient. Respectively it was reckoned with reduced replication of the vector following published hypotheses. Contrary to the expectations, experiments have proven that the vector with these sequences is superior in replication.

These stuffer sequences are linked with the cis elements of a certain serotype, the left ITR and packaging signal on the one side and the right ITR on the other side. This enables replication and packaging of helper-dependent viruses supported by the proteins encoded by a helper of this serotype in a cell line which is permissive for this serotype. A combination of the packaging signal and the ITRs of adenovirus type 5 corresponding to nt 1-450 from sequence NC001406 (NCBI) and a combination of sequence No. 3 containing ITR and packaging signal of adenovirus 11 at the one end and the same sequence from nt 1- 132 sequence (No. 3) at the other end are given as examples.

The invention further encompasses a viral vector based on the sequence of the human serotype 11 adenovirus containing inverted terminal repeats and a packaging signal of a virus from a different serotype preferably ITRs and packaging signal originating from a group C adenovirus, more preferably those of type 5 adenovirus. The vector may be deleted in certain regions to such extent that independent replication without trans-complementation by factors, the genes of which were inactivated by deletion, is impossible and the recombinant viruses are deleted in one or several reading frames of the E1 region and preferably in other reading frames of the E2 and/or E4 region.

According to the invention the vector is based on the sequence of the human serotype 11 adenovirus, in particular one containing a heterologous promoter. This promoter is preferably a SV40 promoter which is positioned between packaging signal and the natural position of the protein IX.

The vector according to the invention may also be characterized by stuffer sequences that are inserted to replace certain regions of the genome or the complete genome with the exception of the left and right ITRs and the packaging signal.

The invention also refers plasmid constructs containing components of the viral vector which are suitable for its production.

Further the invention refers to a cell line which is infectible by the human adenovirus serotype 11 and complements deletions in the viral vector according to claims 1-7. It comprises an adenovirus 11 E1B 55k or a functional homologue thereof as an independent expression unit with a promoter being separate from E1b 19k. The cell line may be derived from HEK 293.

Further the viral vector according to this invention is characterized by the fact that it allows the propagation of a viral vector as a helper virus and the helper virus being characterized by the fact that the packaging signal of the virus is flanked by sequences of site-specific recombinases and that it is inactivated in a cell line complementing adenovirus functions. Human sequences are used as foreign sequences, which exist consequently, interrupted or inverted and intron sequences are used as stuffer sequences for more than 80%. Stuffer sequences are completely or partly extracted from the region of the X chromosome from X152941900-X152976000 and/or chrX149493805-149526200.

Further the invention refers to a therapeutic or a vaccine containing a viral vector according to claims 1-6 or 14-16.

The invented vaccination procedure covers administrating a viral vector to a person to be vaccinated according to claims 1-6 or 14-16.

The features of the invention arise from the elements of the claims and the description and individual features and several features in combined form represent favourable version for which protection is applied with this paper. The combination consists of known (viral vectors, adenoviruses deleted in some or all reading frames) and new elements (heterologous ITRs, promoters and stuffer sequences) influencing each other and enabling the favourable production of recombinant viruses.

The invention leads to deleted adenoviral vectors of the human subtype 11 including all adenoviral vectors being (completely) deleted in some or all reading frames. The invented vectors contain the adenovirus type 11 sequence in the recombinant form, combined with heterologous sequences. These viruses are deleted to such an extent in genome regions that independent replication is impossible without trans-complementation by suitable factors. The invented vector is further characterized by the fact that they
are deleted in the reading frames of the E1 region
contain artificial promoter sequences for transcription
are additionally deleted in the gene of the DNA polymerase
are deleted in the E4 region
   stuffer sequences are inserted as a replacement for certain genome regions or the complete genome with exception of the left and right ITRs and the packaging signal. Foreign sequences comprise sequences of human origin which are used as contiguous, interrupted or inverted sequence. Suffer sequence comprises more intron sequences more than 80% inton sequence and these sequences are completely or partly extracted from the region of the X chromosome from X152941900-X152976000 or chrX149493805-149526200.

The invented viral vector is amplified in a cell line complementing adenoviral gene functions. The cell line contains genes of the E1 region of Ad11 as a continuous sequence with heterologous promoter before E1A and heterologous poly A signal behind the stop codon of E1B55k. The cell line is characterized by the fact that it produces Ad11 E1B 55k or a functional homologue of it as an independent expression unit with own promoter and being separate from E1B 19k.
it expresses additionally genes of the E2 region of Ad11
it expresses additionally genes of the E2 region of Ad2 or 5
it expresses additionally one or more genes of the E4 region of Ad11.

The helper virus represents a deleted adenovirus being essential for the replication in regions such as E1. It carries a heterologous ITR and packaging signal. Additionally the packaging signal of the virus may be flanked by sequences of site-specific recombinases. It is additionally inactivated in this case by a complementary cell line of an adenovirus function carrying the respective recombinase. The helper virus is further characterized by
recombinase sites from recombinases of the integrase type
mutated recombinase sites of recombinases flp and cre, the halfsites of which are only recognized in the original, but not in the recombined form
inverted recombinase sites.

According to the invention the viral vector in the form of a simple, multiple or maximally deleted serotype 11 adenovirus is used as a therapeutic or vaccine. The invention is explained by examples for the execution, but is not limited to these examples.

### EMBODIMENT EXAMPLES

### Example 1

### Cloning of the Type 11 Adenovirus in a Plasmid with the Possibility of Releasing a Viral Vector

Ad11 obtained from American Type Culture Collection (VR-12) was amplified on from Hep-G2 cells and purified using Cs gradient. Virus DNA was isolated by means of pronase treatment, phenol extraction and ethanol precipitation. The DNA was treated with 4 N NaOH for 60 min at 37° C. renaturated and reprecipitated with 0.4 N NaCl, 3 M NaCl, 0.5 M Tris 7.5 for 6 hours at 65° C. and 12 hours at room temperature. The DNA is completely deproteinized. It was treated with Klenow polymerase, cut with Hind III and the fragment mixture was cloned between Hinc II and Hind III sites of pUC18.

A 1.3 kb fragment was found in several clones. This fragment is homologous to known sequences of ITR and the packaging signal of adenovirus 11. It is completely identical to the one of type 5 adenovirus in the terminal 22nt CATCATCAATAATATACCTTAT. In order to obtain the 3′ end, a primer flanked with the terminal sequence of a PacI site Ad11ITRF CTTAATTAACATCATCAATAATATC and primer 11-S2 with the sequence GCTCCGTGCGACTGCTGTTT selected from the fiber region of virus gene bank LO8232 was used. Ad11ITRF contains a single base deletion TAATATAC against the wt sequence caused by a sequencing error. A 2,B kb fragment was amplified and cloned. It contains the 3′ end of the virus. The sequence is identical with the 5′ end of nt 1-132, thus the ITR is 132 bp long. A 472 bp fragment of the 5′ end, flanked by PacI and the internal SnaBI location and thus the packaging signal completely enclosed was rescued and cloned in such way that the shuttle vector for homologous recombination of the virus DNA into a plasmid is produced. As a result of recombination in E. coli BJ 5153 a plasmid of about 15 kb emerged frequently containing the duplicated virus left end. An internal, naturally existing reverted sequence could be the reason. Only in one case a plasmid of 37.5 kb emerged (pAd11 wt) releasing internal Hind III fragments of 14 kb, 5.7 kb, 5.1 kb, 3.3 kb, 2.5 kb, 1.3 kb, 0.7 kb, 0.3 kb.

Only a fragment of 35 kb length is able to cause a cytopathic effect after 16 days when the plasmid is digested with PacI and Ca-transfected into 293 cells. It was found that the digestion yielded two big viral fragments. It means, that a an internal PacI is located in the virus genome. This complicates the production of recombinant viruses. Release of the viral genome from the plasmid vector however is a prerequisite for virus rescue. Therefore plasmid pAd11wt was cut using NcoI and a 4600 bp fragment was religated. From the ligation product a 2000 bp fragment was amplified using a single primer Ad11ITR-F-Pme (sequence ACCGGTTTAAACATCATCAATAATATACCTTAT) and cloned in a KanR minimal vector to yield pAd11Nco. This primer contains a PmeI site. Following PvuII digestion, a gfp encoding fragment containing a unique NotI site was cloned in pAd11Nco and the resulting vector was recombined with pAd11wt after NcoI digestion. The resulting plasmid pAd11wtgfp, flanked by PmeI sites, contains a complete Ad11 genome with a gfp gene cloned in E1 orientation at the end of the packaging signal in position 459 from the left virus end. This plasmid served as a provider for the Ad11 sequences. Parts of the protein IX region and the terminal region of E1B55k was sequenced with a primer which was selected using sequence homologues in the protein IX between the serotypes 7, 5 and 17.

### Example 2

### Cloning of Vectors with Heterologous ITR and/or Packaging Signals

Further Ad11 wt was digested with EcoRV within the plasmid vector and with AfeI in the virus and inserted between SnaBI (at the end of the packaging signal) and BgIII sites (pAd11Afegfp). Following recombination with pAd11wtgfp, digested with NotI, pAd11deltaE1gfp emerged. Using shuttle vector pi5p11gfpPme, containing the Ad55′ITR and flanking sequences from nt 1-190, the Ad11 packaging signal from nt 198-440, the Ad5 3′ITR (103 bp), Ad11 fragments of 3361 bp (PIX region) and from (−) 330 to (−)80 of the right Ad11 end (E4 region), the plasmids pAdi5p11wtgfp and pAdi5p11dE1gfp were obtained by recombination with pAd11wtgfp and pAd11dE1gfp respectively. Both plasmids contain Ad5ITRs and Ad11 packaging signal in a Ad11 vector. Further in pi5p11gfpPme the packaging region from 11 was replaced by Ad5 nt1-450. Following recombination with pAd11Iwtgfp and pAd11dE1gfp respectively pAdip5-11 and pAdip5-11dE1gfp were obtained. Both now contain the Ad5 ITRs and the Ad5 packaging signal.

### Example 3

### Cloning of SV40 Promoter and an Ad5 Packaging Signal Flanked by frt Sites in E1 Deleted Vectors

A Hind III fragment containing the SV40 promoter and the protein IX gene region was extracted from pAd11FRT(1-5)SV40 and cloned in the unique Hind III location of pshAd5frt. pshAdip511frt contains the complete Ad5 IRT, followed by a packaging signal flanked by frt sequence (no. 5). The plasmid was recombined with pAdi5p11wtgfp. The resulting vector pHip511frt contains the Ad11 sequence deleted in E1, ITRs and packaging signal of Ad5 with the packaging signal being flanked by frt sites and the SV40 promoter before the reading frame of PXI. In the same way the HindIII fragment from pAd11frt(1-5)SV40 was inserted downstream of gfp.

In order to analyze the effect of this promoter a tk polyadenylising signal was extracted from pgfpN1 as PvuII BspHI and cloned in StuI of pshAdip511frt. This poly A signal is located directly behind the SV40 promoter and blocks its activity as a promoter, however not an indirect effect as an enhancer for other regions of the genome.

### Example 4

### Production of a Complementary Cell Line for E1 Deleted Vectors of Ad11

The coding sequence of Ad11E1B55k without promoter and poly A was amplified using primers Ad1 155kFXhoI AACTCGAGAATGGATCCCGCAGACT and Ad11E1R-KpnI AATGGTACCTTAGTCAGTTTCTTCTCCAC at template pAd11wi and was cloned to KpnI and XhoI Verdau into the vector phPGKLgfp. E1A is controlled by the human PGK promoter and the polyadenylising signal of E1B is replaced by the early poly A signal of SV40. This plasmid was transfected using polyfect (Quiagen) in HEK293 cells and selected for 3 weeks using 400 μg/ml G418. Clones were thinned out by dilution streak in 96 Well plates and thereafter were checked using PCR with the primers Ad1155kF-XhoI and Ad11E1R-KpnI for existence of the gene Ad11E1B55k. Positive clones were transfected with plasmid Ad11deltaE1gfp after linearazing and clones with visible CPE were used for reamplification of the virus for comparison after 9 days. The clone showing the highest number of developed gfp-induced units D7 was cryoconserved and applied as a helper cell line.

### Example 5

### Production of Recombinant Subtype 11 Viruses by Transfection

2 μg each of the plasmids containing adenoviral vectors were linearized with PmL and transfected with effectene (Quiagen) as per instruction of the manufacturer in 293 Klon D7 (Ad1155k exprimating) or in 293 cells in 2 Wells each of a 6 Well plate. Every day the cells were checked for cytopathic effects. For vectors with the gfp gene the Well was simultaneously checked for expansion of the gfp expression.

| | | Transfection | Transfection |
|---|---|---|---|
| Vector | Description | in 293 | in 293 D7 |
| pAd11wt | wild type, all viral | cytopathic | cytopathic |
| | sequences of Ad11 | effect after | effect after |
| | | 6 days | 6 days |
| pAdi5p11wtgfp | without deletion, Ad5 | cytopathic | cytopathic |
| | ITRs Ad11 packaging | effect after | effect after |
| | signal, gfp | 6 days | 7 days |
| pAdip5-11wtgfp | without deletion, Ad5 | no virus | no virus |
| | ITRs packaging signal, | developed | developed |
| | gfp | | |
| PAd11dE1gfp | complete E1 deletion, all | no virus | cytopathic |
| | viral sequences of Ad11 | developed | effect after |
| | | | 8 days |
| pAdi5p11dE1gfp | complete E1 deletion, | no virus | no virus |
| | Ad5ITRs Ad11 packag- | developed | developed |
| | ing signal, gfp controlled | | |
| | by CMV promoter | | |
| pAdip5-11dE1gfp | complete E1 deletion, | no virus | cytopathic |
| | Ad5ITRs and packaging | developed | effect after |
| | signal, gfp controlled by | | 7 days |
| | CMV promoter | | |
| pAdip5-11dE1 | complete E1 deletion, | no virus | cytopathic |
| SV40gfP | Ad5ITRs and packaging | developed | effect after |
| | signal followed by SV40 | | 5 days |
| | promoter, gfp controlled | | |
| | by CMV promoter | | |
| pHip511frt | complete E1 deletion, | no virus | cytopathic |
| | Ad5ITRs and packaging | developed | effect after |
| | signal with frt sites | | 4 days |
| | followed by SV40 | | |
| | promoter | | |
| pHip511frtPA | complete E1 deletion, | no virus | cytopathic |
| | Ad51TRs and packaging | developed | effect after |
| | signal with frt sites | | 5 days |
| | followed by SV40 | | |
| | promoter and poly A | | |
| | signal | | |

Surprisingly the wild type as well as E1 deleted viruses with heterologous ITRs and autologous packaging signal are not propagable. However, it appears that a complete exchange of ITRs and packaging signal by elements of Ad5 does not provoke considerable slowdown of virus replication. Existence of a SV40 promoter between packaging signal and protein IX shortens the time to a complete cytopathic effect. A poly A signal following the promoter has no influence on this effect.

All viable viruses were rescued and used for the infection of a second passage in suitable cell substrate (293 or 293D7). Viruses of this passage were used for the production of passage 3 and resulting viruses were tested in a plaque assay. Here the effect of the described elements was confirmed.

Hip511frt and Ad11de1gfp were purified from 293KlonD7 (20 Ø15 cm-cell culture dishes each) via CsCl step- and continuous gradients. For Hip511frt a 20 times stronger upper band (empty virus capsids) and a weak lower band (complete virus) was obtained. To the contrary, in Ad11de1gfp a clear lower band and a weak upper band were noticed. This confirms the hypothesis that although Hip511frt is well amplifyable in 293KlonD7, it produces a surplus of empty capsids due to ineffective packaging.

### Example 6

### Cloning of Vector PHCA

Genomic DNA was isolated from 15 ml full blood of a test person by means of SDS lysis followed by sodium chloride precipitation and ethanol precipitation. 100 ng each were used as template in PCR reactions with Taq polymerase (expand long template kit, Roche). The following primers were used:

| | |
|---|---|
| 152-1 | CTTCGAATGAACCTGGAGTTGACTT |
| | and |
| 152-2 | CTTAATTAACCTCACCTGGCCAACATC |
| | for fragment 1 |
| 152-3 | CATCGATCGGCACGCTGTTGATT |
| | and |
| 152-4 | CTTCGAACCTGCTCTGTGAAGCACTG |
| | for fragment 2 |
| 152-5 | ACGGCCGAAGGATTACATGAGCTTAG |
| | and |
| 152-6 | CATCGATCAGGCTTGGCTTCTCT |
| | for fragment 3. |

First, fragment 3 was cloned into vector pPCR4blunttopo (Invitrogene). To construct a high capacity vector a shuttle vector was made containing ITR and packaging signals of the type 5 adenovirus. For this purpose the adenovirus 5 sequence of nt1-nt443 flanked by PmeI at 5′ and EcoR52I as well as Hind II at 3′ ends and the sequence 35520-35935 flanked by Hind III and EcoR52I at 5′ end and PmeI at 3′ end by PCR (expand high fidelity PCR kit, Roche) were amplified. Thereafter, both fragments were co-amplified with external primers by annealing in the overlapping region and cloned into the EcoRV location of the vector pAC. Then a polylinker with the sequence GGCCGGATATCGATATCTTCGAACGGTTAATTA was inserted.

Fragment 3 was directly cloned into this polylinker between CIaI and Eco521 and thus pHCA3 was established. Control digests revealed the lack of a SacI site in fragment 3 in contrast to the genomic sequence.

For cloning of fragments 1 and 2, 300 bp and 400 bp regions from the fragment ends were amplified, linked by overlapping primers using PCR and cloned after digestion with PacI and BstBI as flank 1, and after digestion with ClaI and BstBI as flank 2 into HCA3. Flanks 1 and 2 each contain unique NotI and SaII sites. The resulting vector was linearized with NotI and used for homologous recombination with the PCR fragment 1 in E. coli RecA+RecBC-sbcBC-. In the same way after linearizing with SaII fragment 2 was inserted. The resulting vector has unique BstBI and CIaI at the borders of fragments 1 and 2, and 2 and 3 respectively. For both locations shuttle vectors were established containing 300 bp at both sides of the unique locations. A β-galaktosidase gene and DsRed 1 (red fluorescence protein, ClonTech) controlled by the RSV promoter was cloned under the control of the CMV promoter and was inserted by recombination. PHCAredfp and a control vector A carrying the gfp gene under the control of the CMV promoter were separated by restriction digest from the plasmid backbone and 2 μg each together with 2 μg helper virus genome released from the plasmid were co-transfected in 293 cells using calcium phosphate precipitation. After complete infection of the culture the lysate was harvested and 293 cells were infected again. The persentage of cells with green and red florescence was evaluated in the following passages. It was found that in passage 3 about 15% of the cells were red fluorescent, but only 7% green. Thus the vector pHCA is superior to the control vector in its amplification rate as a result of replication and packaging.

Further the following is stated about the vector system of the human type 11 adenovirus:

It is known the region E1A proteins provoke the change of the cell cycle in combination with stimulation of the virus replication. They are also activating the transcription, however do not bind themselves on their own to DNA, but interact with cellular factors. The expert also knows that E1A of a serotype may complement the vector of a different serotype. As we know this does not refer to E1 B, because it interacts with other viral proteins, especially with E4orf6. Therefore, it is not possible to amplify the invented vector in cell line 293, which expresses the E1 region of Ad5. The E1B region codes for 2 independent proteins of 19k and 55k, which are coded by a common mRNA. The promoter of E1B is located in the E1A region. Since 19k of Ad5 is present in 293 cells and no serotype specificity is expected based on its function to inhibit apoptosis, it was chosen to express E1B55k in HEK293 cells as an individual gene controlled by heterologous poly A and promoter.

The cell line as per claim 9 is characterized by expressing adenovirus 11E1B55k.

### SEQUENCES

Sequence No.1 (Sequence between Ad11 E1B55k Stopkodon and pIX
Startkodon)
GGTGAGTATTGGGAAAACTTTGGGGTGGGATTTTCAGATGGACAGATT
GAGTAAAAATTTGTTTTTTCTGTCTTGCAGCTGTC
Sequence No.2 Ad11 55k reading frame
ATGGATCCCGCAGACTCATTTCAGCAGGGGATACGTTTTGGATTTCAT
AGCCACAGCATTGTGGAGAACATGGAAGGTTCGCAAGATGAGGACAA
TCTTAGGTTACTGGCCAGTGCAGCCTTTGGGTGTAGCGGGAATCCTGA
GGCATCCACCGGTCATGCCAGCGGTTCTGGAGGAGGAACAGCAAGAG
GACAACCCGAGAGCCGGCCTGGACCCTCCAGTGGAGGAGGCGGAGTA
GCTGACTTGTCTCCTGAACTGCAACGGGTGCTTACTGGATCTACGTCC
ACTGGACGGGATAGGGGCGTTAAGAGGGAGAGGGCATCTAGTGGTAC
TGATGCTAGATCTGAGTTGGCTTTAAGTTTAATGAGTCGCAGACGTCC
TGAAACCATTTGGTGGCATGAGGTTCAGAAAGAGGGAAGGGATGAAG
TTTCTGTATTGCAGGAGAAATATTCACTGGAACAGGTGAAAACATGTT
GGTTGGAGCCTGAGGATGATTGGGAGGTGGCCATTAAAAATTATGCCA
AGATAGCTTTGAGGCCTGATAAACAGTATAAGATTACTAGACGGATTA
ATATCCGGAATGCTTGTTACATATCTGGAAATGGGGCTGAGGTGGTAA
TAGATACTCAAGACAAGGCAGTTATTAGATGCTGCATGATGGATATGT
GGCCTGGGGTAGTCGGTATGGAAGCAGTAACTTTTGTAAATGTTAAGT
TTAGGGGAGATGGTTATAATGGAATAGTGTTTATGGCCAATACCAAAC
TTATATTGCATGGTTGTAGCTTTTTTGGTTTCAACAATACCTGTGTAGA
TGCCTGGGGACAGGTTAGTGTACGGGGATGTAGTTTCTATGCGTGTTG
GATTGCCACAGCTGGCAGAACCAAGAGTCAATTGTCTCTGAAGAAATG
CATATTTCAAAGATGTAACCTGGGCATTCTGAATGAAGGCGAAGCAAG
GGTCCGCCACTGCGCTTCTACAGATACTGGATGTTTTATTTTGATTAAG
GGAAATGCCAGCGTAAAGCATAACATGATTTGCGGTGCTTCCGATGAG
AGGCCTTATCAAATGCTCACTTGTGCTGGTGGGCATTGTAATATGCTG
GCTACTGTGCATATTGTTTCCCATCAACGCAAAAAATGGCCTGTTTTTG
ATCACAATGTGATGACGAAGTGTACCATGCATGCAGGTGGGCGTAGA
GGAATGTTTATGCCTTACCAGTGTAACATGAATCATGTGAAAGTGTTG
TTGGAACCAGATGCCTTTTCCAGAATGAGCCTAACAGGAATTTTTGAC
ATGAACATGCAAATCTGGAAGATCCTGAGGTATGATGATACGAGATCG
AGGGTACGCGCATGCGAATGCGGAGGCAAGCATGCCAGGTTCCAGCC
GGTGTGTGTAGATGTGACTGAAGATCTCAGACCGGATCATTTGGTTAT
TGCCCGCACTGGAGCAGAGTTCGGATCCAGTGGAGAAGAAACTGACT
AA
Sequence No. 3 (ITR and packaging region of Ad11)
CATCATCAATAATATACCTTATAGATGGAATGGTGCCAATATGTAAAT
GAGGTGATTTTAAAAAGTGTGGATCGTGTGGTGATTGGCTGTGGGGTT
AACGGCTAAAAGGGGCGGTGCGACCGTGGGAAAATGACGTTTTGTGG
GGGTGGAGTTTTTTTGCAAGTTGTCGCGGGAAATGTGACGCATAAAAA
GGCTTTTTTCTCACGGAACTACTTAGTTTTCCCACGGTATTTAACAGGA
AATGAGGTAGTTTTGACCGGATGCAAGTGAAAATTGTTGATTTTCGCG
CGAAAACTGAATGAGGAAGTGTTTTTCTGAATAATGTGGTATTTATGG
CAGGGTGGAGTATTTGTTCAGGGCCAGGTAGACTTTGACCCATTACGT
GGAGGTTTCGATTACCGTGTTTTTTACCTGAATTTCCGCGTACCGTGTC
AAAGTCTTCTGTTTTTAC
Sequence No. 4 (SV40 promoter and A11 protein IX region)
CAGCTGTGGAATGTGTGTCAGTTAGGGTGTGGAAAGTCCCCAGGCTCC
CCAGCAGGCAGAAGTATGCAAAGCATGCATCTCAATTAGTCAGCAAC
CAGGTGTGGAAAGTCCCCAGGCTCCCCAGCAGGCAGAAGTATGCAAA
GCATGCATCTCAATTAGTCAGCAACCATAGTCCCGCCCCTAACTCCGC
CCATCCCGCCCCTAACTCCGCCCAGTTCCGCCCATTCTCCGCCCCATGG
CTGACTAATTTTTTTTATTTATGCAGAGGCCGAGGCCGCCTCGGCCTCT
GAGCTATTCCAGAAGTAGTGAGGAGGCTTTTTTGGAGGCCTAGGCTTT
TGCAAAAAGCTTCACGCTGCCGCAAGCACGAGCTCGCTGTCATGAGTG
GAAACGCTTCTTTTAAGGGGGGAGTCTTCAGCCCTTATCTGACAGGGC
GTATCCCATCCTGGGCAGGAGTTCGTCAGAATGTTATGGGATCTACTG
TGGATGGAAGACCCGTCCAACCCGCCAATTCTTCAACGCTGACCTATG
CTACTTTAAGTTCTTCACCTTTGGACGCAGCTGCAGCTGCCGCCGCCGC
TTCTGTTGCCGCTAACACTGTGCTTGGAATGGGTTACTATGGAAGCAT
CATGGCTAATTCCACTTCCTCTAATAACCCTTCTACCCTGACTCAGGAC
AAGTTACTTGTCCTTTTGGCCCAGCTGGAGGCTTTGACCCAACGTCTGG
GTGAACTTTCTCAGCAGGTGGTCGAGTTGCGAGTACAAACTGAGTCTG
CTGTCGGCACGGCAAAGTCTAAATAA
Sequence No. 5 (Adenovirus 5 ITR with packaging signal flanked of 2 Frt sites)
CATCATCAATAATATACCTTATTTTGGATTGAAGCCAATATGATAATG
AGGGGGTGGAGTTTGTGACGTGGCGCGGGGCGTGGGAACGGGGCGGG
TGACGTAGTAGTGTGGCGGAAGTGTGATGTTGCAAGTGTGGCGGAACA
CATGTAAGCGACGGATGTGGCAAAAGTGACGTTTTTGGTGTGCGGAAG
TTCCTATTCTCTAGAAAGTATAGGAACTTCGGTACCGGTGTACACAGG
AAGTGACAATTTTCGCGCGGTTTTAGGCGGATGTTGTAGTAAATTTGG
GCGTAACCGAGTAAGATTTGGCCATTTTCGCGGGAAAACTGAATAAGA
GGAAGTGAAATCTGAATAATTTTGTGTTACTCATAGCGCGTAATCTCT
AGCATCGATGAAGTTCCTATTCTCTAGAAAGTATAGGAACTTCG

List of abbreviations

| | |
|---|---|
| Ad/AdV | Adenovirus |
| Ad2, 5, or 11 | Adenovirus serotype |
| bp | Base pairs |
| cDNA | to mRNA complementary DNA |
| CMV | Cytomegalievirus |
| Cre | Rekombinase of phage P1 |
| dCTP | Cytidintriphosphate |
| E1A, E1B, E3, or E4 | Organizational, subgenomic regions in |
| | AdV genome |
| flp | Rekombinase |
| GC | Amount of desoxyguanosin- and desoxycytidin- |
| | nucleotids in the DNA |
| i.m. | intramuscular |
| ITR | Inverted Terminal Repeats |
| NF1, NF2, NF3 | cellular factors, which are concerned in |
| | AdV-replication |
| PCR | Polymerase chain reaction |
| pIX, pIVa2 | adenoviral proteins |
| RSV | Rous Sarkom Virus Promoter |
| SDS | Natriumdodecylsulfate |
| TATA-Box | Transcription factor binding location of the |
| | TATA binding protein |
| Tet | Tetracycline |
| pTP | Terminal protein |

### REFERENCES

Alemany, R., Dai, Y., Lou, Y. C., Sethi, E., Prokopenko, E., Josephs, S. F., and Zhang, W. W. (1997). “Complementation of helper-dependent adenoviral vectors: size effects and titer fluctuations.” J Virol Methods, 68(2), 147-59.
Chen, H. H., Mack, L. M., Kelly, R., Ontell, M., Kochanek, S., and Clemens, P. R. (1997). “Persistence in muscle of an adenoviral vector that lacks all viral genes.” Proc Natl Acad Sci USA, 94(5), 1645-50.
Fisher, K. J., Choi, H., Burda, J., Chen, S. J., and Wilson, J. M. (1996). “Recombinant adenovirus deleted of all viral genes for gene therapy of cystic fibrosis.” Virology, 217(1), 11-22.
Hardy, S., Kitamura, M., Harris-Stansil, T., Dai, Y., and Phipps, M. L. (1997). “Construction of adenovirus vectors through Cre-lox recombination.” J Virol, 71(3), 1842-9.
Kochanek, S., Clemens, P. R., Mitani, K., Chen, H. H., Chan, S., and Caskey, C.

T. (1996). “A new adenoviral vector: Replacement of all viral coding sequences with 28 kb of DNA independently expressing both full-length dystrophin and beta-galactosidase.” Proc Natl Acad Sci USA, 93(12), 5731-6.
Kumar-Singh, R., and Chamberlain, J. S. (1996). “Encapsidated adenovirus minichromosomes allow delivery and expression of a 14 kb dystrophin cDNA to muscle cells.” Hum Mol Genet, 5(7), 913-21.
Mitani, K., Graham, F. L., Caskey, C. T., and Kochanek, S. (1995). “Rescue, propagation, and partial purification of a helper virus-dependent adenovirus vector.” Proc Natl Acad Sci USA, 92(9), 3854-8.
Parks, R. J., Chen, L., Anton, M., Sankar, U., Rudnicki, M. A., and Graham, F. L. (1996). “A helper-dependent adenovirus vector system: removal of helper virus by Cre-mediated excision of the viral packaging signal.” Proc Natl Acad Sci USA, 93(24), 13565-70.
Sandig, V., Youil, R., Bett, A., J., Franlin, L., Oshima, M., Maione, D., Wang, Metzker, M. L., Savino, R., and Caskey, C. (2000). “Optimization of the Helper Dependent Adenovirus System for Production and Potency in vivo.” Proc Natl Acad Sci USA, 1;97(3):1002-7.

1. Viral vector, comprising sequence of human serotype 11 adenovirus containing inverted terminal repeats and packaging signal of a virus of a different serotype.

2. Viral vector of claim 1, wherein said virus from which both the inverted terminal repeats and the packaging signal originate is the same group C adenovirus.

3. Viral vector of claim 1 or 2, comprising deletions in genomic regions incompatible with independent replication in the absence of trans-complementation by factors coding for the deleted genes.

4. Viral vector of claim 1 or 2, comprising a deletion of at least one reading frame from the E1 region.

5. Viral vector, comprising sequence of the human serotype 11 adenovirus containing a heterologous promoter.

6. Viral vector of claim 5 or 21, wherein the heterologous promoter is SV40 promoter and/or promoter being located between packaging signal and natural position of protein IX.

7. Viral vector of of claim 1 or 2 for producing recombinant viruses, comprising a replacement of predetermined genome regions or the whole genome with the exception of the left and right and packaging signals by stuffer sequences.

8. Vector constructs comprising components of the viral vector according to claim 1 or 2 or being suitable for its production.

9. Cell line infectible by a human adenovirus of serotype 11 and being able to complement deletions in a viral vector comprising sequence of human serotype 11 adenovirus containing inverted terminal repeats and packaging signal of a virus of a different serotype.

10. Cell line of claim 9, expressing 11 E1B 55k or a functional homologue thereof as an independent expression unit separate from E1B 19k with its own promoter.

11. Cell line of claim 9 or 10, derived from HEK 293.

12. Viral vector of claim 1 or 2, as a helper virus for enabling propagation of a viral vector for producing recombinant viruses, comprising sequence of human serotype 11 adenovirus containing inverted terminal repeats and packaging signal of a virus of a different serotype, and a replacement of predetermined regions or the whole genome with the left and right packaging signals by stuffer sequences.

13. Virus of claim 12, as a helper virus, wherein packaging signal of the virus is flanked by recognition sequences of site-specific recombinases and it is inactivated in a complementing cell line containing respective recombinase, and the cell line being infectible by a human adenovirus of serotype 11 and being able to complement deletion in a viral vector comprising sequence of human adenovirus containing inverted terminal repeats and packaging signal of a virus of a different serotype.

14. Viral vector of claim 7, wherein the stuffer sequences comprise continuous, interrupted or inverted human sequences.

15. Viral vector of claim 7, wherein the stuffer sequences are comprised of more than 80% intronic sequences.

16. Viral vector of claim 7, wherein the stuffer sequences are completely or partly extracted from the region of the X chromosome from X152941900-X152976000 and/or from chromosome X149493805-149526200.

17. Therapeutic or vaccine comprising a viral vector, the viral vector comprising sequence of human serotype 11 adenovirus containing inverted terminal repeats and packaging signal of a virus of a different serotype.

18. (Canceled)

19. Viral vector of claim 2, wherein said group C adenovirus is adenovirus type 5.

20. Viral vector of claim 4, comprising deletions of reading frames of the E2 and/or E4 regions.

21. Viral vector of claim 1, comprising a heterologous promoter.

22. Method of treatment, comprising vaccination with a vaccine according to claim 17.


SEQUENCE LISTING (18 sequences)

SEQ ID NO: 1 - DNA, 83 bp, Adenovirus subtype 11
ggtgagtattgggaaaactttggggtgggattttcagatggacagattgagtaaaaatttgttttttctgtcttgcagctgtc

SEQ ID NO: 2 - DNA, 1485 bp, Adenovirus subtype 11
atggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatctagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagcctgaggatgattgggaggtggccattaaaaattatgccaagatagctttgaggcctgataaacagtataagattactagacggattaatatccggaatgcttgttacatatctggaaatggggctgaggtggtaatagatactcaagacaaggcagttattagatgctgcatgatggatatgtggcctggggtagtcggtatggaagcagtaacttttgtaaatgttaagtttaggggagatggttataatggaatagtgtttatggccaataccaaacttatattgcatggttgtagcttttttggtttcaacaatacctgtgtagatgcctggggacaggttagtgtacggggatgtagtttctatgcgtgttggattgccacagctggcagaaccaagagtcaattgtctctgaagaaatgcatatttcaaagatgtaacctgggcattctgaatgaaggcgaagcaagggtccgccactgcgcttctacagatactggatgttttattttgattaagggaaatgccagcgtaaagcataacatgatttgcggtgcttccgatgagaggccttatcaaatgctcacttgtgctggtgggcattgtaatatgctggctactgtgcatattgtttcccatcaacgcaaaaaatggcctgtttttgatcacaatgtgatgacgaagtgtaccatgcatgcaggtgggcgtagaggaatgtttatgccttaccagtgtaacatgaatcatgtgaaagtgttgttggaaccagatgccttttccagaatgagcctaacaggaatttttgacatgaacatgcaaatctggaagatcctgaggtatgatgatacgagatcgagggtacgcgcatgcgaatgcggaggcaagcatgccaggttccagccggtgtgtgtagatgtgactgaagatctcagaccggatcatttggttattgcccgcactggagcagagttcggatccagtggagaagaaactgactaa

SEQ ID NO: 3 - DNA, 451 bp, Adenovirus subtype 11
catcatcaataatataccttatagatggaatggtgccaatatgtaaatgaggtgattttaaaaagtgtggatcgtgtggtgattggctgtggggttaacggctaaaaggggcggtgcgaccgtgggaaaatgacgttttgtgggggtggagtttttttgcaagttgtcgcgggaaatgtgacgcataaaaaggcttttttctcacggaactacttagttttcccacggtatttaacaggaaatgaggtagttttgaccggatgcaagtgaaaattgttgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttac

SEQ ID NO: 4 - DNA, 797 bp, Adenovirus subtype 11
cagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcacgagctcgctgtcatgagtggaaacgcttcttttaaggggggagtcttcagcccttatctgacagggcgtatcccatcctgggcaggagttcgtcagaatgttatgggatctactgtggatggaagacccgtccaacccgccaattcttcaacgctgacctatgctactttaagttcttcacctttggacgcagctgcagctgccgccgccgcttctgttgccgctaacactgtgcttggaatgggttactatggaagcatcatggctaattccacttcctctaataacccttctaccctgactcaggacaagttacttgtccttttggcccagctggaggctttgacccaacgtctgggtgaactttctcagcaggtggtcgagttgcgagtacaaactgagtctgctgtcggcacggcaaagtctaaataa

SEQ ID NO: 5 - DNA, 427 bp, Adenovirus subtype 11
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcggaagttcctattctctagaaagtataggaacttcggtaccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgatgaagttcctattctctagaaagtataggaacttcg

SEQ ID NO: 6 - DNA, 22 bp, Adenovirus subtype 11
catcatcaataatataccttat

SEQ ID NO: 7 - DNA, 25 bp, Artificial Sequence
  Description of Artificial Sequence Primer
cttaattaacatcatcaataatatc

SEQ ID NO: 8 - DNA, 20 bp, Artificial Sequence
  Description of Artificial Sequence Primer
gctccgtgcgactgctgttt

SEQ ID NO: 9 - DNA, 33 bp, Artificial Sequence
  Description of Artificial Sequence Primer
accggtttaaacatcatcaataatataccttat

SEQ ID NO: 10 - DNA, 25 bp, Artificial Sequence
  Description of Artificial Sequence Primer
aactcgagaatggatcccgcagact

SEQ ID NO: 11 - DNA, 29 bp, Artificial Sequence
  Description of Artificial Sequence Primer
aatggtaccttagtcagtttcttctccac

SEQ ID NO: 12 - DNA, 25 bp, Artificial Sequence
  Description of Artificial Sequence Primer
cttcgaatgaacctggagttgactt

SEQ ID NO: 13 - DNA, 27 bp, Artificial Sequence
  Description of Artificial Sequence Primer
cttaattaacctcacctggccaacatc

SEQ ID NO: 14 - DNA, 23 bp, Artificial Sequence
  Description of Artificial Sequence Primer
catcgatcggcacgctgttgatt

SEQ ID NO: 15 - DNA, 26 bp, Artificial Sequence
  Description of Artificial Sequence Primer
cttcgaacctgctctgtgaagcactg

SEQ ID NO: 16 - DNA, 26 bp, Artificial Sequence
  Description of Artificial Sequence Primer
acggccgaaggattacatgagcttag

SEQ ID NO: 17 - DNA, 23 bp, Artificial Sequence
  Description of Artificial Sequence Primer
catcgatcaggcttggcttctct

SEQ ID NO: 18 - DNA, 33 bp, Artificial Sequence
  Description of Artificial Sequence Polylinker
ggccggatatcgatatcttcgaacggttaatta